(19) United States
(12) Patent Application Publication
(10) Pub. No.: US 2010/0099195 A1
(43) Pub. Date: Apr. 22, 2010

(54) Zinc-Based Screening Test and Kit for Early Diagnosis of Prostate Cancer

(75) Inventor: Christopher Frederickson, Galveston, TX (US)

Correspondence Address: Don D. Cha, 547 Buena Vista Road, Golden, CO, 80401, US

(73) Assignee: ANDRO DIAGNOSTICS, Galveston, TX (US)

(21) Appl. No.: 12/528,447

(22) PCT Filed: Jan. 25, 2008
(86) PCT No.: PCT/US08/52123

§ 371 Date: Aug. 24, 2009

(30) Foreign Application Priority Data
Jan. 25, 2007 (US) 11698229
May 15, 2007 (US) 11803478

(51) Int. Cl. G01N 33/20; G01N 21/00; C12M 1/00

(52) U.S. Cl. 436/81; 422/82.05; 435/288.7

(57) ABSTRACT

The present invention provides devices, kits, and methods for determining the zinc level in a fluid sample of a subject. Such determination is useful in screening for the presence of or the increased risk of having prostate cancer.

### FIELD OF THE INVENTION

The present invention relates generally to devices, kits, and methods for determining the zinc level in a fluid sample of a subject.

### BACKGROUND OF THE INVENTION

Zinc serves many functions in animals. For example, zinc is an essential micronutrient, a component of enzymes and other proteins, and it is also an ionic signal. Zn²⁺ moves through gated membrane channels and among various organelles and storage depots within cells modulating protein function by binding to and detaching from zinc-dependent proteins throughout the cell.

Like calcium, excess free zinc in body tissues is toxic. Zn²⁺ is selectively stored in, and released from, the presynaptic vesicles of a specific type of neuron, which is found chiefly in the mammalian cerebral cortex. These zinc-releasing neurons also release glutamate, and the term ‘gluzinergic’ has, therefore, been proposed to describe them. Most glutamate- and zinc-releasing neurons have their cell bodies in either the cerebral cortex or the limbic structures (amygdala and septum) of the forebrain. Therefore, the glutamate and zinc-releasing neuronal system comprises a vast cortical—limbic associational network that unites limbic and cerebrocortical functions.

Since the discovery of zinc's signaling role, a broad outline of the function of glutamate- and zinc-releasing neurons has emerged. Without being bound by any theory, it is believed that zinc modulates the overall excitability of the brain through its effects on glutamate, and γ-aminobutyric acid (GABA), receptors. It is also believed to be important in synaptic plasticity.

More recently, it has been observed that the level of zinc in semen falls, for example, by 50%-90%, in the early stages of prostate cancer while not changing in benign hypertrophy. Changes in total zinc levels as measured by atomic absorption spectroscopy (AAS) or X-ray fluorescence (XRF) have been associated with prostate cancer.

At least two major disadvantages with above observation have prevented clinical use of these observations. Most of the previous studies required a biopsy to measure total zinc levels. This does not provide a particular advantage to the patient as pathological analysis of the specimen serves as the gold standard despite the 10 to 20% false negative rate. Biopsies are time and resource intensive and carry their own morbidity rate. Second, total zinc measurements using AAS is impractical due to equipment size/cost and the requirement of skilled operators. Hence, measuring zinc in complex biological matrices, such as semen and determining the sizes of the different “pools” of zinc and the changes if any in these multiple zinc pools is a daunting bioanalytic problem. Thus the literature on zinc and prostate cancer is alarmingly error ridden. For example estimates in the scientific literature of total zinc in prostate tissues and total zinc in semen vary over a range of nearly 100 fold. In some instances, total zinc levels can be decreased in a patient due to a decrease in zinc carrier protein or from prostate cancer. Such fluctuation further reduces the accuracy and utility of using the total zinc level as a prostate cancer screening test.

It has been shown that free zinc, i.e., the fraction of zinc that is not protein bound, in semen becomes bound to various protein within a relatively short period of time, thereby reducing the reliability of the zinc level depending on the length of time between obtaining the sample and determining the level of zinc. For example, it has been shown that the prostate gland secretes approximately 10 mM of zinc into prostatic fluid. However, it also secretes about 100 mM of citrate and forms Zn₂Cit₃ (5 mM) in prostatic fluid. The binding of zinc to citrate is relatively weak, with the binding affinity being in the 10-50 micromolar, range. Therefore, when Zn₂Cit₃ is present at millimolar concentrations, there is always a substantial concentration (approximately 1 micromolar) of Zn²⁺ present, and the exchange of the Zn²⁺ with the zinc in citrate is ongoing and rapid. Once the prostatic fluid mixes with the fluid from the seminal vesicles and from the testes, the Zn₂Cit₃ is distributed into about three fold greater volume and the Zn₂Cit₃ is therefore diluted to about 5/3 mM. In addition, at the time of the mixing, some of the zinc is separated from the Zn₂Cit₃ and becomes bound more tightly to other peptides and proteins in the seminal plasma. And as the time passes, the amount of free zinc in the semen sample decreases. Thus, the level of free zinc in semen will vary significantly depending on the amount of time lapsed between obtaining the sample and conducting the test. Moreover, in general after about 1 hour at room temperature, the amount of free zinc in semen samples becomes almost undetectable; thereby, rendering free zinc level test at an off-site facility virtually impracticable.

Accordingly, there is a need for a more accurate method for detecting free zinc level in a subject.

### SUMMARY OF THE INVENTION

Some aspects of the invention provide a method for screening a subject for the presence or elevated risk of developing prostate cancer comprising:

measuring a level of free zinc in a seminal or prostatic sample of the subject; and

comparing the measured zinc level with a control zinc level to screen the subject for the presence or elevated risk of developing prostate cancer,

wherein when the seminal sample of the subject is used, said step of measuring the level of free zinc comprises:

subjecting the sample to the free zinc level measuring step within 5 minutes or less of the time the sample leaves the subject's body; or

using the measured level of free zinc to determine the level of free zinc at the time the sample leaves the subject's body.

In some embodiments, the sample comprises prostatic fluid.

In some embodiments, the control zinc level comprises the level of free zinc in the normal individual.

Yet in other embodiments, the level of free zinc in the fluid is measured optically. Within these embodiments, in some cases the level of free zinc is measured visually. For example, by comparing the color or fluorescence of the sample with a reference chart.

In other embodiments, a reagent that is capable of releasing zinc from citrate is added to the sample prior to the step of measuring the free zinc level.

Still in other embodiments, the method of measuring the free zinc optically comprises:

contacting the sample to a zinc-binding moiety under conditions sufficient to bind the free zinc to the zinc-binding moiety, wherein the zinc-binding molecule has a different optical property when bound to zinc relative to its non-zinc bound state;

determining the optical property of the zinc-binding molecule; and

correlating the optical property of the zinc-binding molecule with the level of free zinc in the fluid.

In many embodiments, the zinc-binding moiety comprises a chromophore or a fluorophore.

Other aspects of the invention provide a method for determining the presence or elevated risk of developing prostate cancer in a subject, said method comprising:

determining a level of free zinc in a seminal or prostatic sample of the subject; and

correlating the level of free zinc to the presence of prostate cancer or elevated risk of developing prostate cancer in the subject,

wherein when the seminal sample of the subject is used, said step of determining the free zinc level comprises:

subjecting the sample to the free zinc level determination process within 5 minutes or less of the time the sample leaves the subject's body; or

using the determined level of free zinc to determine the level of free zinc at the time the sample leaves the subject's body.

In some embodiments, the level of free zinc is determined optically. Within these embodiments, in some cases the level of free zinc is measured visually, for example, by comparing the color or fluorescence of the sample with a reference chart.

Yet in other embodiments, the method of determining the level of free zinc comprises:

contacting the sample to a zinc-binding molecule under conditions sufficient to bind the free zinc to the zinc-binding moiety, wherein the zinc-binding molecule has a different optical property when bound to zinc relative to its non-zinc bound state; and

correlating the optical property of the zinc-binding molecule to the level of free zinc in the sample.

In some embodiments, the zinc-binding molecule comprises a chromophore or a fluorophore.

Yet other aspects of the invention provide a device for visually determining a zinc level in a bodily fluid, said device comprising:

a zinc-binding molecule that allows optical determination of the zinc level in a bodily fluid sample;

means for confining the zinc-binding molecule to a region in space; and

a surface effective for visually observing optical change of said zinc-binding molecule within the region thereby allowing optical determination of the zinc level.

In some embodiments, the device allows determination of the level of free zinc optically. Within these embodiments, in some cases the level of free zinc is measured visually, for example, by comparing the color or fluorescence of the sample with a reference chart.

Still in other embodiments, the device further comprises a reagent that releases zinc from a protein in said bodily fluid. Within these embodiments, in some instances the reagent is a pH lowering reagent, diethyl pyrocarbonate, cystine diethyl pyrocarbonate residue, a protease, a zinc-chelating reagent with zinc binding affinity of at least 1 mM, or a combination thereof.

Yet in other embodiments, the zinc-binding molecule has a different optical property when bound to zinc relative to its non-zinc bound state.

In other embodiments, the zinc-binding molecule is confined to the defined region via covalent binding to a solid substrate.

Still yet in other embodiments, the zinc-binding molecule is retained in the defined region due to the partition co-efficient of the molecule.

In still other embodiments, the device further comprises an interface that separates a bodily fluid collection region from the zinc-binding molecule. Within these embodiments, in some instances, the interface allows permeation of zinc ions, often selectively, to reach the region containing the zinc-binding molecule. Within these instances, in some cases, the selective permeation is due to size, solubility, charge, or other physical properties.

Yet other aspects of the invention provide a kit for determining the zinc level in a bodily fluid of an individual comprising a device disclosed herein and a reference chart.

In some embodiments, the kit further comprises a container for collecting the bodily fluid.

Still in other embodiments, the reference chart is a zinc level color chart. Within these embodiments, the zinc level color chart designates a specific color for low, normal and high levels of zinc.

Other aspects of the invention provide a method of optically determining a zinc level in a bodily fluid of an individual comprising:

contacting the bodily fluid sample obtained from the individual with a zinc-binding molecule, wherein the zinc-binding molecule has a different optical property when bound to zinc relative to its non-zinc bound state; and

observing the optical property of the zinc-binding molecule, thereby determining the zinc level in the bodily fluid of the individual.

In some embodiments, the zinc-binding molecule is bound to a solid substrate.

Still in other embodiments, zinc is separated from at least a portion of the bodily fluid sample prior to contacting with the zinc-binding molecule.

In other embodiments, the optical property of the zinc-binding molecule is chromophore or fluorophore.

Yet in other embodiments, the step of determining the zinc level comprises visually comparing the optical property of the zinc-binding molecule with a reference chart.

The invention also provides a method for screening an individual at risk for prostate cancer. The method comprises obtaining a sample of a zinc-containing fluid from the individual and measuring the level of free zinc and/or zinc bound to endogenous ligands in the sample. The zinc level(s) from the at risk individual are compared with zinc levels found in a normal individual known not to have prostate cancer where a decreased zinc level in the at-risk individual compared to the level of free zinc in the normal individual correlates to a risk of developing prostate cancer, thereby screening the individual.

The invention can also include a further method step of measuring the total protein in the sample. In this method, the zinc level can be a ratio of the free zinc to the total protein, a ratio of the bound zinc to the total protein, or a ratio of free zinc plus bound zinc to the total protein.

The invention also is directed to a method for screening an individual at risk for prostate cancer comprising obtaining a sample of prostate secretions in a fluid from the individual and measuring a level of free zinc in the fluid sample. In some embodiments, the level of free zinc from the at risk individual is compared with a level of free zinc in a normal individual that does not have prostate cancer. A decreased level of free zinc in the at-risk individual compared to the level of free zinc in the normal individual correlates to a risk of developing prostate cancer, thereby screening the individual.

The prostate secretions can be a fluid comprising seminal plasma of ejaculate. In such embodiments, in many instances the step of obtaining the sample to be analyzed can further include separating large globular proteins and prostasomes from the seminal plasma including free zinc, for example, via size-exclusion and/or column fractionation or via antibody- or aptamer-binding thereto. In an alternative related method the prostate secretions can be prostatic fluid where the sample obtaining step includes massaging the prostate to advance the prostatic fluid comprising the prostate secretions and collecting a post prostatic massage prostatic fluid.

### BRIEF DESCRIPTION OF THE DRAWINGS

FIG. 1A is a graph showing fluorescence intensity at various zinc levels using carbonic anhydrase (CA) as the zinc-binding molecule and either ABDN or dansylamide as the fluorescent reporter.

FIG. 1B is a graph showing ratiometric shift in fluorescence anisotropy of apoCA zinc.

FIG. 1C is a graph shows two different mutants of carbonic anhydrase having different binding kinetics (and have different affinities) for zinc. The fluorescence indicates zinc binding by ABDN.

FIG. 2 shows a plot of protein concentration in various fractions in men presenting symptoms of prostatitis or prostate enlargement or malfunction. Two clear peaks are shown; the first, HMW, peak contains prostasomes and is identified as the “prostasomal peak”.

FIG. 3A is a graph showing decrease in the free zinc level in two men with prostate tumors relative to normal. The lines with range bars depict average results for 15 cancer-free men (±SD).

FIG. 3B is a graph showing decrease in the free zinc concentration (top) and protein concentration (bottom) in two men with prostate tumors relative to normal. The lines with range bars depict average results for 15 cancer-free men (±SD). Zinc in the prostasomal fractions (#15-20) was reduced from Abs=0.71 to Abs=0.32 in the two men with adenocarcinoma.

FIG. 4 shows plot of serum PSA (top) and free zinc (bottom) levels among men 40-80 years old.

FIG. 5 is a schematic representation of free and bound zinc in the three fluids that comprise ejaculate.

FIG. 6 is a bar graph showing frequency distribution of the total zinc in the seminal plasma of 18 normal men.

FIG. 7 is a bar graph representation of free and bound zinc in prostatic fluid (massage expressed) in 6 men.

FIG. 8 is a graph showing “free” (weakly bound) zinc in successive protein fractions of seminal plasma.

FIG. 9 shows one embodiment of a device of the invention for determining the zinc level in a sample.

FIG. 10 is a graph showing that the free zinc level is lower in the expressed prostatic fluid from men with cancer than from men with normal or benignly enlarged prostates.

FIG. 11 is a graph showing that the concentration of free zinc in prostatic fluid had no obvious relationship to the volume of tumors that were found in the glands at histology.

FIG. 12 is a plot of the amount of free zinc in prostatic fluid from men with confirmed adenocarcinoma and men with no known cancer.

FIG. 13 shows one embodiment of the device for detecting zinc level in a fluid sample.

### DETAILED DESCRIPTION OF THE INVENTION

As used herein, the term “subject” refers to any recipient of the prostate cancer screening as discussed herein. Typically, the subject is a mammal, and often the subject is a human.

As used herein, the term “free zinc” refers to rapidly exchangeable zinc which is that concentration of zinc that will bind to saturation to a zinc-binding sensor molecule having moderate affinity (dissociation constant, K_{D}, of about 1 or higher) and a diffusion-limited on-rate within a brief epoch, e.g., 1 min, after mixing. Thus, “free zinc” is the zinc one can “see” with a colorimetric, voltametric, or fluorescent sensor within 1 minute. Thus, the term “free” is defined by the off-rate of the ligand with which the zinc is associated prior to measuring. If the zinc is Zn²⁺ coordinated with Cl⁻ or acetic acid, the “off rate” is virtually instantaneous. With weak-binding organic ligands, such as citrate (K_{D} of about 5 nM), or glutamate (K_{D} of about 6), the off rates are still rapid (msec to sec), but for tightly-binding ligands, such as carbonic anhydrase, the time for one-half of the zinc to come off spontaneously is about 2 years.

It is estimated that prostate cancer kills about 40,000 men in the United States each year and there are approximately 330,000 new cases diagnosed annually. In men, prostate cancer is second only to lung cancer in mortality. Castration, treatment with anti-androgens, and prostatectomy with its associated urogenital risk, are all treatments that seriously compromise the quality of male life.

Monitoring the health or function of the prostate gland on matters such as the presence of adenocarcinoma or benign prostate hypertrophy (BPH) by measuring analytes such as proteins or peptides in the urine or blood is an established art, with the protein “PSA” being the most commonly used analyte. Measuring serum prostate-specific antigen (PSA), a serine protease, level and prostate digital rectal exams are currently the only early diagnostic tests in routine use to screen for prostate cancer. However, small, aggressive tumors can be missed by digital rectal exams and even by needle biopsy, and only modest increases in prostate-specific antigen, i.e., below the 4 ng/mL thresh hold between normal and elevated PSA levels, are generated by these tumors. These aggressive tumors have the potential to suddenly dedifferentiate and grow, spread, and metastasize rapidly.

In addition to such lethal false negatives, false positives also plague the PSA test, causing unnecessary tests and medical expense and distress to patients. In some studies, it has been shown that among men above 50 years old, an age group of men most susceptible to prostate cancer, 80% of those having PSA test levels above 4 ng/mL will turn out to not have prostate cancer. Thus, there is a need for a prostate cancer screen with improved ability to differentiate between prostate cancer and benign conditions such as prostatitis, benign prostatic hypertrophy (BPH) or enlarged prostate, inflammation and infection, and to differentiate between slow-growing and fast-growing cancers.

More dangerous results are those tests that show false negative. Consider the patient who suffers a false negative, for example, in which a small tumor, e.g., T1a,b, T2a, is missed by digital rectal exams and missed in needle biopsy, even an ultrasound-guided, 6-sector biopsy, and does not raise the serum PSA to alarming levels, i.e., PSA below 4. Depending on the grade of tumor, a patient with a Gleason Pattern GP 4-5 tumor could have a metastatic disease with poor prognosis within a year whereas a patient with a GP 1-2 tumor might experience little changes in a year. Since most prostate cancers are slow-growing, there is a clear need for a routine diagnostic screen that can pick up prostate cancer before it is large enough to produce symptoms.

Zinc is the most ubiquitous heavy metal in the human body. In the male reproductive system, semen has 3 mM zinc, which is approximately 200-1000 fold more than those found in saliva, tears, vaginal secretions, urine or blood. Spermatozoa are richly endowed with zinc both in their cytosol and on their exterior. Without being bound by any theory, it is believed that the source of zinc is in part from the testes, which concentrates zinc in and on the spermatozoa, and in part from the secretory cells lining the ducts of the lateral lobes of the prostate gland. At the fine and ultrastructural level, the zinc in the prostate tubules is concentrated at the apical ends of the secretory cells, in the interstities between the cells, and in the lumen of the seminal ducts.

Physiologically, the epithelial secretory cells show relatively high velocity uptake of zinc that is driven by testosterone. Thus, it is believed that the epithelial secretory cells take up zinc, sequester it in secretory granules, and secrete the contents of the granules into the lumen, thereby generating the high zinc content of the semen.

Again without being bound by any theory, it is believed that the prostate gland has a uniquely high zinc content which is localized to the lateral lobes and that the prostate loses from 50% to 90% of that zinc in prostate cancer. In contrast, the zinc levels increase in benign prostatic hypertrophy (BPH) and show no consistent change in prostatitis.

Since most of the zinc in the prostate is concentrated in the lumen and secretory surfaces of the seminal tubules, e.g., in the secretory fluids, the observed drop of 50-90% in total zinc content would be expected to require a significant drop in the zinc content of the seminal fluid. In fact, it has been shown that patients with stage T3-T4 tumors showed a 95% decrease in zinc in ejaculate, and patients with palpable tumors showed an 84% decrease in zinc in post-prostatic massage fluid. In benign prostatic hypertrophy, the zinc level was found to be either unchanged or increased. Thus, the amount of zinc concentrated in the gland, secreted into the prostatic fluid, and (therefore) appearing in the ejaculate is markedly decreased in adenocarcinoma of the prostate, but not in BPH.

In some aspects, methods of the invention can detect even the small, nonpalpable tumors, e.g., T1a-c, T2a, that generate only modest increases in serum PSA, i.e., below 4 ng/mL, but have the potential to dedifferentiate rapidly to Gleason pattern 4-5 and thus grow and metastasize rapidly. In fact, the present inventor has shown that the zinc level is a sensitive and selective cancer indicator.

Still other aspects of the invention provide methods for detecting free zinc level in a fluid sample of a subject. In some embodiments, methods of the invention determines the free zinc level by subjecting the sample to the free zinc level measuring step within 5 minutes or less of the time the sample leaves the subject's body; or using the measured level of free zinc to determine the level of free zinc at the time the sample leaves the subject's body. Without being bound by any theory, it has been shown by the present inventors, see the Examples section below, that the amount of free zinc in the seminal fluid obtained from a subject decreases upon standing as the free zinc becomes bound by what is believed to be various proteins and/or other zinc binding moieties that are present in the semen.

The rate at which free zinc becomes bound by proteins and/or other zinc binding moieties that are present in the semen depends on a variety of factors including, but not limited to, the amount of proteins and/or other zinc binding moieties present in the semen, temperature at which the semen is kept, as well as other factors. In general, however, it has been found by the present inventor that at room temperature a relatively accurate determination of the free zinc level can be achieved in the semen sample, when the sample is subjected to a free zinc determination process within 15 minutes, typically within 10 minutes, often within 5 minutes, more often within 3 minutes, and still more often within 1 minute of the sample leaving the subject's body.

In some embodiments, one can obtain a relatively accurate determination of the free zinc level at the time the sample leaves the subject's body even if the semen sample is subjected to the free zinc level determination step after the given time above. It has been found by the present inventor that the rate at which free zinc becomes bound can be measured. This allows one to extrapolate the level of free zinc at the time the sample leaves the subject's body (i.e., time zero) by knowing the amount of time it took between the sample leaving the subject's body, e.g., via ejaculation, and when the sample was subjected to the free zinc level determination process. While some measurements of the free zinc level in the semen at various times after ejaculation is shown in the Examples section, one skilled in the art can obtain individual tailored free zinc decreasing rate by following the processes described herein. Moreover, as more and more data are gathered (either from the particular individual undergoing the test and/or from general population), one can combine these data to more accurately extrapolate the free zinc level at time zero.

In other aspects of the invention, a prostatic fluid is used as the sample. Unlike the seminal fluid, it has been found by the present inventor that the free zinc level in the prostatic fluid does not vary significantly over time. Accordingly, use of the prostatic fluid does not require one to subject the sample to the free zinc level determination process or extrapolation to time zero. Thus, when the prostatic fluid is used to determine the free zinc level, methods of the invention allow one to determine the free zinc level at an off-site facility or at other convenient time and/or facility without the need for extrapolation.

In some embodiments, the free zinc level is used to distinguish between a decrease in zinc carrier protein or from prostate cancer. In general, the free zinc fraction of the subject is more specifically affected by cancerous changes of the prostate relative to the decrease in zinc carrier protein.

As stated above, the prostate gland secretes zinc and citrate and forms Zn₂Cit₃ in prostatic fluid. During ejaculation, the prostatic fluid mixes with the fluids from the seminal vesicles and from the testes. And at the time of the mixing, some of the zinc is separated from the Zn₂Cit₃ and becomes bound more tightly to other peptides and proteins in the seminal plasma. The result is that some of the zinc becomes associated with the prostatsomal proteins or prostatsomes (globular protein complexes).

It is believed that when the prostate gland becomes cancerous and the secretory cells of the prostate dedifferentiate, they cease to secrete the zinc-citrate, and the zinc in the prostatic fluid falls dramatically. Therefore, the amount of zinc in the two prostate-derived factions (the “prostatsomal” fraction and the “zinc citrate” fraction) falls selectively and specifically while the amount of zinc associated with other components (e.g., spermatazooa) does not decline. Hence, in some embodiments, the level of free zinc in the prostasomal fraction, the zinc citrate fraction, the seminal plasma fraction, or a combination thereof is determined to assess the prostate status of the subject.

In some aspects of the invention, the level and/or speciation of zinc in semen or prostatic fluid is used. Accordingly, many aspects of the invention provide a zinc-based diagnostic kit for prostate cancer. In many embodiments of the invention, zinc that is bound to citrate is released from the citrate prior to determining the zinc level. There are many metal ions which has higher affinity for citrate than zinc, for example, calcium, magnesium, etc. Suitable metal ions that can cause release of zinc in zinc-citrate complex can be readily determined, for example, by comparing the dissociation constant between zinc-citrate and metal-citrate. By adding such metal ions (more appropriately the metal ion source, e.g., metal salts), to the sample, for example, as an aqueous solution, one can facilitate determination of the free zinc level.

Still in other aspects of the invention, methods for determining the semen and/or prostatic fluid zinc levels can be used alone or combined with serum PSA levels as a diagnosis for prostate cancer. Some kits of the present invention can be used for routine testing of seminal or prostatic zinc in the clinic or at home.

It is believed that the fall in semen zinc at the onset of prostate cancer is not equally specific to the different semen zinc pools, i.e., free zinc, zinc bound to endogenous ligands, such as microligand bound zinc, small protein bound zinc, large protein bound zinc, and/or spermatozoan zinc. Accordingly, some embodiments of the invention provide methods for screening for prostate cancer by determining the free zinc level. Still other embodiments of the invention provide methods for determining the level of free zinc, zinc bound to endogenous ligands, zinc bound to small proteins, zinc bound to large proteins, or a combination thereof.

Some devices, kits and methods of the invention can be used to determine the distribution, speciation and concentrations of zinc in prostate tissue and seminal fluid, e.g., ejaculate or the post-prostate massage expressed prostatic fluid. Still other embodiments of the invention provide methods for determining free versus bound zinc in seminal plasma or prostatic fluid; ligand binding, e.g., speciation, of zinc in semen; free versus bound zinc in prostate tissue; zinc concentrations in individual spermatozoa; and/or histochemical localization(s) of the free stainable zinc. Within these embodiments, in some instances Timm-Danscher fluorescence and/or Synchrotron X-ray fluorescence can be used to determine the zinc level.

Using the present invention, the means, ranges, and variances of zinc contents in prostate tissue, prostatic fluid and ejaculate can be determined in men with or without, as a control, prostate cancer. Typically, in normal prostates (i.e., absence of prostate cancer) free zinc concentration in prostatic fluids, as measured fluorimetrically in prostatic fluid diluted by 1:2000 in HEPES at pH 7.4, is about 7 mM (as referred to the undiluted prostatic fluid) or higher, often about 8 mM or higher, and more often about 9 mM or higher. In some instances, free zinc level in prostatic fluids of about 4 mM or less, often about 2 mM or less, and more often about 0.5 mM or less is indicative of the presence of prostate cancer or a higher risk for the presence of prostate cancer.

In seminal fluids, the free zinc level (within approximately 15 minutes of the sample leaving the subject's body—i.e., ejaculation) of about 7/3 mM or higher, often about 8/3 mM or higher, and more often about 9/3 or higher mM is indicative of normal prostate. In some instances, free zinc level in seminal fluids of about 4/3 mM or less, often about 2/3 mM or less, and more often about 0.5/3 mM or less is indicative of the presence of prostate cancer or a higher risk for the presence of prostate cancer.

The present invention also allows determination of: 1) free and total zinc in whole seminal fluid or ejaculate; 2) free and total zinc in seminal plasma; 3) free and total zinc in prostatic fluid; 4) zinc bound to specific subsets of seminal proteins; 5) zinc bound to citrate; and 6) zinc concentration in individual spermatozoa. Some embodiments of the invention provide methods for screening for prostate cancer by determining the free zinc level in a semen sample and/or prostatic sample of a subject. Generally, any statistically significant decrease in the zinc level compared to those found in normal individual is indicative that the subject is at risk of developing prostate cancer or has prostate cancer. The samples that are useful for screening prostate cancer include, but are not limited to, whole seminal fluid, seminal plasma, expressed prostatic fluid, spermatozoa, cytosol of spermatozoa, seminal globulin protein, and a combination thereof.

In some instances, methods of the invention include determining free” or “rapidly-exchangeable” zinc level in the semen; the level of zinc bound to organic ligands in the semen, such as proteins, peptides, amino acids, and/or small molecules; and the zinc level in cells, such as spermatozoa and/or endothelial cells that have sloughed into the semen. Exemplary methods for determining the zinc level include fluorimetric and colorimetric methods in which the amount of fluorescence or light absorbance, respectively, is visually observed or determined using a detector. It should be appreciated that while some embodiment determine the zinc level visually, the present invention is not limited to these techniques. In general any colorimetric, fluorimetric, as well as any other optical or non-optical methods that allow determination of different concentrations of the zinc level can be used. Some embodiments allow determination of the zinc level in and/or on spermatozoa. For example, spermatozoa can be stained and the free zinc level can be determined fluorimetrically using Znpyr and TSQ. Alternatively, the free zinc level in and/or on spermatozoa can be determined using AMG or EM.

Other embodiments of the invention use the subject's prostatic fluid for determining the zinc level. Any methods for obtaining prostatic fluids can be used. For example, prostate secretions can be obtained by prostate massage to channel or advance the prostatic fluid to the urethra and collecting it therefrom. In some instances, the prostatic fluid is collected in a first volume of urine produced post massage. Alternatively, upon further prostate massage, the prostatic fluid that emerges from the uretha can be collected and used to determine the zinc level. The free zinc can be determined by any of the methods known to one skilled in the art including those described herein such as colorimetric and/or fluorimetric methods.

Free zinc or total zinc, including bound zinc released as free zinc, whether seminal or prostatic, can be measured optically by exposing a free zinc-containing fluid to a chromophore or fluorophore in a colorimetric, absorptionmetric or fluorimetric assay. Zinc-binding moieties present on the fluorophores or chromophores, such as, but not limited to, quinoline, BAPTA, ethylene diamine tetra acetic acid (EDTA), pyridine, TPEN, P.A.R., 8-hydroxy quinoline, Eriochrome black, Alloxan tetrahydrate, Arsenazo III, Calconcarboxylic acid, Calmagite, Chromeazuro 1 1,5-Diphenylcarbazide, Diphenylcarbazone, Dithizone, Eriochrome Black, Hydroxynaphthol blue, Methylthymol Blue, 1-(2-Pyridylazo)-2-naphthol, Pyrocatechol Violet, 5-Sulfosalicylic acid dehydrate, Tiron, Zincon, and 2-(5-Bromo-2-pyridylazo)-5-(N-propyl-N-sulfopropylamino)phenol (5-Br-PAPS) bind free zinc from the fluid. Upon illumination, the amount of light absorbed by the chromophore or emitted by the fluorophore positively correlates to the amount of free zinc in the fluid. Chromophores, such as dithizone, zincon, 4-(2-pyridylazo) resorcinol or other molecule that changes absorptive properties upon binding zinc, and fluorophores, such as fluorescein, rhodamine, allexa, or dansylamide, are well known in the art and commercially available.

In one particular embodiment, a fluorophore is mixed with the zinc-containing prostatic fluid. In some instances, the fluorophore is attached to a solid substrate surface, such as a glass slide, a capillary tube, a metal, a solid polymer, a ceramic, as well as any other solid substrates known to one skilled in the art that are useful in conducting assays. The sample is contacted with the solid substrate under conditions sufficient to allow binding of any free zinc that may be present in the sample with the zinc-binding molecule or moiety. The attached fluorophore can then be excited with an evanescent wave of light and emitted light (i.e., fluorescence) is used to determine the free zinc level. Alternatively, a sensor can be positioned on the surface that is opposite to the surface exposed to the prostatic fluid to detect emitted light by to determine the free zinc level.

In some embodiments, the fluorescent methods allow for quantitation, or at least a relative quantitation, as they are typically stoichiometric or ratiometric, e.g., with the apoCA. Accordingly, some embodiments of the present invention determine the zinc level by fluorescence analysis. In some cases, different fluorescence methods are available based on the subcellular location of the zinc level to be determined. For example, the membrane impermeable apoCA is generally not suitable for determining the zinc level in vesicles, and the “trappable” Newport green, which is metabolized in cytosol, is generally not suitable for determining the zinc level in the cytosol. In contrast, the lipophilic stains TSQ or Zinpyr can be used to determine the zinc level in intracellular organelles, cytosol, and in extracellular fluid.

Free and total zinc in solution can be measured by any of the various methods known to one skilled in the art including, but not limited to, apoCA fluorimetric method and stable isotope dilution mass spectrometry. In some cases, microspectrofluorimetric methods or silver staining autometalography can be used to measured zinc that is not in solution. Thus, extracellular zinc, such as zinc on the outer surfaces of spermatozoa or zinc loosely coordinated with globular proteins, can be stained with cell-impermeable stains such as Newport Green, and the fluorescein-based metal sensors Zinpyr or Zin-naphthopyr (ZNP), or by TSQ. Exemplary Zinpyrs include ZP-4 and ZP-8, which are disclosed in U.S. Patent Application Publication No. 20020106697.

In some aspects of the invention, the zinc level screening method is combined with the PSA assay. Such a combination increases the accuracy of prostate cancer test. For example, results of decreased levels of zinc combined with increased levels of PSA compared to those found in normal individual provide more sensitive and accurate prostate cancer screening as well as providing corroboration of test results.

Other aspects of the invention include diagnostic kits that can be used to screen for prostate cancer. In some embodiments, such kits include determining the zinc level via a colorimetric or a ratiometric fluorimetric measurement system. In some cases, such kits use LED and/or CCD's which can aid in determining the zinc level. Determination of the zinc level can be performed in a clinic for measuring the clinically-appropriate “pool” of semen or prostatic fluid zinc or it can be conveniently performed at home using the kit that allows determination of the zinc level in whole seminal fluid.

In some embodiments, the kit can be used to determine the zinc level in one or more pools of free zinc, bound zinc or zinc in cells, as disclosed herein. Diagnosis can be based on the relative abundance of zinc in these pools and typically depends on which of these pools sizes or ratios of zinc abundance in different pools is the most accurate predictor of nascent prostate cancer.

In some embodiments, determining the zinc level comprise separating the free zinc from the whole semen. Such separation can be achieved by, for example, dialysis or any other methods known to one skilled in the art for separating ions or small molecules from other components in a sample. Polymeric membranes (e.g., dialysis membranes) with pore size of 100 MW allow zinc to diffuse through the membrane while preventing other larger molecules such as fluorescent probes for zinc from diffusing through. Such membranes allow separation of zinc from biological fluids as well as keeping other molecules such as fluorescent probes for zinc from passing through the membranes.

In many embodiments, the kit for determining free zinc level comprises a fluorescent probe for zinc that is placed on one side of a zinc separation material, such as a polymeric membrane or a molecular sieve. The sample, such as semen from the subject, is placed on the other side of the zinc separation material. In using such a kit, the sample is provided with a sufficient time to allow the zinc to diffuse through the separation material and bind to the fluorescent probe.

In some cases, the sample is treated with a detergent, e.g. triton-X 100, to lyse the membranes of prostasomes to release the zinc that is sequestered in secretory prostasomes.

Many probes that are useful in determining the presence zinc are known in the art. Exemplary zinc probes include, but are not limited to, apoCA+ a reporter, such as dansylamide or ABDN, a Zinpyr dye or stain, such as ZP-1, ZP-4 and ZP-8, and a zin-napthopyr, such as ZNP-1, TSQ, Fluo-zinc, and coumazin. Others probes that are suitable for the present invention are well known to one skilled in the art and are readily available.

When determining the level of the bound zinc, typically the bound zinc is separated from the binding molecule prior to determining the zinc level. To separate zinc from zinc-binding organic molecules, standard separation methods familiar to those skilled in the art are used including, but not limited to, chromatography, gel separation and antibody-based extraction/purification. It should be appreciated that not all zinc-binding ligands need to be identified or purified to determine the zinc level.

An immobilized antibody or aptamer can be used to trap the zinc-binding ligand of interest on a substrate. Washing the resulting substrate then removes the non-selected molecules and vehicle from the substrate. Determining the zinc level in the isolated zinc-binding ligands can be achieved by releasing captured zinc from the ligand using any of the methods known to one skilled in the art including, but not limited to, chemically treating the ligand with a chemical agent such as nitric oxide, hydrogen peroxide or weak acid, or other chemicals that release the zinc from organic ligands, which are well known to those skilled in the art. Some of the chemical agents denature the zinc-binding ligand, thereby the zinc to be released into the surrounding fluid. The level of zinc is then determined by the methods described herein including fluorimetric or colorimetric methods. Accordingly, in some embodiment the kit includes a zinc-binding molecule (e.g., an antibody) immobilized on an appropriate substrate surface to separate zinc-binding ligands.

When desired, determining the total zinc level in cells is achieved by separating the cells from the seminal plasma, e.g., by filtration. The separated cells are lysed (e.g., by triton X, as described), and the bound zinc is released using any of the known methods including those described herein. The resulting mixture is then analyzed to determine the level of zinc.

In a kit form, methods of the invention can be accomplished on simple, home test formats similar to those utilized for measuring various analytes, e.g., glucose, cholesterol, or drugs of abuse, in bodily fluids, such as saliva, serum or urine. Methods for at home antibody separation technique similar to those used in home pregnancy tests can be used. Some kits of the invention also include colorimetric tests to determine the zinc level. Colorimetric tests for at-home analysis are well known and include home-test kits for glucose, cholesterol, ketone, and other analytes. Some kits include filtration system. Filtration system for at-home test are also well known and include in home-tests for glucose test.

Some kits of the invention comprise a “ZnDectec” cassette, a pouch comprising a dialysis bag, a small digital reader and a chart. In some embodiments, the “ZnDectec” cassette is a 4-5 cm container comprising a mixture of carbonic anhydrase (apoCA) and a reporter molecule, such as dansylamide (DNSA), or others that are well known to one skilled in the art including those disclosed herein. In using this kit, a seminal fluid sample is placed into the pouch that is designed to fit into the cassette. The free zinc ions in the sample pouch moves out of the pouch and into the detection cassette where the zinc ions become to apoCA and form the holoCA-dansylamide complex.

In some cases the pouch, which is substantially depleted of free zinc ions, is then removed from the cassette. The level of zinc is then determined by fluorescence, for example, by placing the cassette into a simple fluorescence reader having excitation and emission filters set to collect the fluorescence of the holoCA-dansylamide complex. In some instances, the fluorescence reader is used to convert the fluorescence values to values of zinc levels. An individual can check the chart included in the kit against the values of zinc levels obtained and determine whether the measured zinc levels fall into, for example, one of three ranges: normal, pre-disposition to prostate cancer and prostate cancer.

The level of zinc can also be used as a basis for differential imaging of healthy versus cancerous prostate tissue. There are many non-toxic or benign zinc binding compounds, including, but not limited to, citrate, histidine, and diethyldithiocarbamate (such as those used in Antabuse, and clioquinol which is a USP antimicrobial), that can be taken orally and reach the prostate tissue. To image zinc, a molecule or agent that undergoes a change or shift in a parameter like infrared light absorption or NMR frequency upon binding zinc is used. Such a zinc contrast agent allows imaging of the prostate, for example, by optoacoustic imaging or MRI. NMR contrast agents for zinc are well known to one skilled in the art. See, for example, Benters et al., J. Biochem., 1997, 322, 793-799. The prostate can also be imaged using ⁶⁹Zn or ⁷²Zn isotopes.

Some aspects of the invention provide a method for screening an individual at risk for prostate cancer. Such method generally comprises obtaining a sample of a zinc-containing fluid from the individual; measuring a level of one or both of free zinc and zinc bound to endogenous ligands in the sample; comparing the zinc level(s) from the at risk individual with zinc levels found in a control sample (e.g., normal individual known not to have prostate cancer or individual known to have prostate cancer); and correlating the zinc level in the at-risk individual compared to the zinc level in the control sample, thereby screening the individual. The zinc level may be the free zinc in the fluid or a ratio of the free zinc to the bound zinc.

In some instances, methods of the invention can also comprise determining the total protein level in the sample. In some cases, the total amount of protein in the sample can be determined by ultraviolet light absorption of the protein in the sample. For example, determination of the zinc level can be a ratio of the free zinc to the total protein, a ratio of the bound zinc to the total protein, or a ratio of free zinc plus bound zinc to the total protein.

The zinc level in the sample can be determined optically. In some embodiments, the zinc level is determined visually. Within these embodiments, in some cases the method comprises contacting the sample to a zinc binding molecule which comprises a chromophore and/or a fluorophore moiety; providing conditions sufficient to allow the zinc in the sample, if present, to bind to the zinc-binding molecule; and determining the zinc level by the amount of light that is either absorbed by the chromophore or emitted by the fluorophore. Typically, such determination include correlating the light absorption or light emission with the zinc level in the sample. Representative examples of a useful chromaphores include, but are not limited to, dithizone, zincon, 4-(2-pyridylazo)resorcinol and other chromaphores that change absorptive properties upon binding zinc. Representative examples of fluorphores include, but are not limited to, fluorescein, rhodamine, allexa, and dansylamide. Representative examples of a zinc-binding moieties include, but are not limited to, quinoline, BAPTA, ethylene diamine tetra acetic acid, pyridine, TPEN, P.A.R., 8-hydroxy quinoline, Eriochrome black, Alloxan tetrahydrate, Arsenazo III, Calconcarboxylic acid, Calmagite, Chromeazuro 1 1,5-Diphenylcarbazide, Diphenylcarbazone, Dithizone, Eriochrome Black, Hydroxynaphthol blue, Methylthymol Blue, Pyrocatechol Violet, 5-Sulfosalicylic acid dehydrate, Tiron, Zincon, and 2-(5-Bromo-2-pyridylazo)-5-(N-propyl-N-sulfopropylamino)phenol (5 -Br-PAPS).

In some embodiments, methods of the invention can also include releasing the zinc bound to endogenous ligands in the sample and determining the zinc level. The zinc level can be determined by any of the methods known to one skilled in the art including those disclosed herein. In some cases, the zinc level is determined electrochemically by correlating the electrochemical property of the sample before and after releasing the bound zinc. In other cases, the total zinc is determined fluorimetrically or absorptiometrically. The released zinc can be separated from the sample using a membrane that is permeable or semi-permeable to zinc. Another method for separating the zinc from the sample include placing the sample on a surface containing carrier or iontophore molecules effective to transport zinc ions across the surface.

In other aspects, methods of the invention include releasing the zinc bound to endogenous ligands in the sample and determining the zinc level before and after releasing the bound zinc. The released zinc can be separated from the sample.

The sample obtained from the subject can be ejaculate, seminal fluid, seminal plasma, prostatic fluid, or a combination thereof. In some embodiments, the sample is prostatic fluid.

In another embodiment of the present invention, there is provided a method for screening an individual at risk for prostate cancer. The method generally comprises obtaining a sample of prostate secretions in a fluid from the individual; measuring a level of free zinc in the fluid sample; comparing the level of free zinc from the at risk individual with a level of free zinc in a normal individual that does not have prostate cancer; and comparing the level of free zinc in the at-risk individual compared to the level of free zinc in the normal individual, thereby screening the individual. The prostate secretions can be in a fluid comprising seminal plasma of ejaculate where the step of obtaining the sample includes separating large globular proteins and prostasomes from the seminal plasma including free zinc via size-exclusion column fractionation. The prostate secretions can also be in a fluid comprising seminal plasma of ejaculate where the step of obtaining comprises separating large globular proteins and prostasomes from the seminal plasma including free zinc via antibody- or aptamer-binding thereto. In some cases, the prostate secretions are in prostatic fluid, and the step of obtaining the sample include massaging the prostate to advance the prostatic fluid comprising the prostate secretions into the urethra and collecting a post prostatic massage prostatic fluid therefrom. The prostatic fluid can be obtained in a first volume of urine produced post prostatic massage. In some cases, prostate can be massaged repeatedly until the prostatic fluid emerges from the urethra.

The zinc level in the prostatic fluid can be determined fluorimetrically as described herein. In some instances, the method includes adding a detergent to the prostatic fluid to lyse and dissociate prostasomes and globular proteins in the prostatic fluid thereby releasing zinc bound thereto. In some instances, the zinc level before and after lysing is determined. In other instances, the prostatic fluid is mixed with the zinc-binding molecule that comprises a fluorophore. Other embodiments of the invention include attaching the fluorophore at a distance no more than 350 nm from a surface of a solid substrate to which the sample is exposed.

Some aspects of the invention include exciting the fluorophore with an evanescent wave of light and detecting the light emissions of the excited fluorophore to determine the zinc level. In some embodiments, a sensor on a surface of the solid substrate is positioned opposite to the surface exposed to the sample to detect fluorescent emissions. In yet other embodiments, methods include separating the sample from the fluorophore via a semipermeable membrane permeable to zinc ions but not permeable to the fluorophore.

Other aspects of the invention provide devices for determining or assessing zinc levels in bodily fluids. Such devices include a reagent that is capable of causing the release of the protein-bound or citrate-bound zinc in said bodily fluid; a zinc-binding molecule; a means of confining the molecule to a defined region in space; an interface bounding one surface of the region; and a surface to allow visual observation of color change of the zinc-binding molecule within the region. In some embodiments, the reagent causing the release of the protein- or citrate-bound zinc is a pH lowering reagent. In other embodiments, the reagent causing the release of the protein-bound zinc is diethyl pyrocarbonate or cystine diethyl pyrocarbonate residue. Still in other embodiments, the reagent causing the release of the protein-bound zinc is a mixture of proteases. In some particular embodiments, the reagent causing the release of the protein-bound zinc is a zinc-chelating reagent binding to zinc with affinities of about 1 mM or higher. Still in other embodiments, the protein-bound zinc is bound to the semenogelins I and II proteins of the semen. In general, the device assesses prostate function by determining the concentration of free zinc in a bodily fluid. Typically, the zinc-binding molecule undergoes a change in optical property (e.g., colorimetric property or fluorimetric property) upon binding with zinc. In some particular embodiments, the zinc-binding molecule is selected from, but not limited to, the group including P.A.R., 8-hydroxy quinoline, Eriochrome black, Alloxan tetrahydrate, Arsenazo III, Calconcarboxylic acid, Calmagite, Chromeazuro 1 1,5-Diphenylcarbazide, Diphenylcarbazone, Dithizone, Eriochrome Black, Hydroxynaphthol blue, Methylthymol Blue, 1-(2-Pyridylazo)-2-naphthol, Pyrocatechol Violet, 5-Sulfosalicylic acid dehydrate, Tiron, Zincon, 2-(5-Bromo-2-pyridylazo)-5-(N-propyl-N-sulfopropylamino)phenol (5-Br-PAPS), and a combination thereof.

In some embodiments, the zinc-binding molecule is confined to a defined region of about 5 nanometers or more but no more than about 10 mm in all 3-axis. In some cases, the zinc-binding molecule is confined to the defined region via covalent binding to a solid substrate. In other cases, the zinc-binding molecule is retained in the defined region due to the partition co-efficient of the molecule. Yet in other cases, the zinc-binding molecule is dissolved in a polar solvent. In such cases, the zinc-binding molecule is typically many-fold more soluble in the polar solvent than the aqueous environment of bodily fluid.

Yet in other embodiments, the interface of the device allows selective permeation of zinc ions to reach the region containing the zinc-binding molecule. Within these embodiments, in some cases, the selective permeation is due to size, solubility, charge, and/or other physical properties. In other cases, the interface comprises a size-exclusion filter. Within these cases, in some instances the size-exclusion filter excludes molecules greater than 0.22 microns in diameter.

Other aspects of the invention provide kits for determining the zinc levels in the bodily fluid sample of an individual. Such kits include a device for determining the zinc level as described herein; and a reference chart. Typically, the reference chart is a zinc color chart that is based on the optical property of a zinc-binding molecule, for example, colorimetric property or fluorimetric property. Often the zinc color chart designates a specific color for low, normal and high levels of zinc. In some embodiments, the kit also includes a container for collecting the bodily fluid.

Still yet other aspects of the invention provide methods for determining a zinc level in the bodily fluid of an individual. Such methods include obtaining the bodily fluid from the individual; releasing the protein-bound zinc in said bodily fluid; contacting the bodily fluid thus obtained with the device for determining the zinc level in bodily fluids; waiting for the color change reaction; and comparing the color change to a reference chart. The release of the protein bound zinc can be accomplished by a pH lowering reagent, diethyl pyrocarbonate, cystine diethyl pyrocarbonate residue, a protease, or a mixture thereof. In other embodiments, the release of the protein bound zinc is accomplished by a zinc-chelating reagent with zinc affinities of about 1 mM or higher. In some embodiments, the reference chart is a zinc color chart as described herein. Typically, the zinc color chart provides a specific color for low, normal and high levels of zinc. Generally a low level of zinc is indicative of prostatic disease such as, but not limited to, benign prostatic hyperplasia or adenocarcinoma of the prostate.

Other aspects of the invention provide methods for determining a zinc level in the ejaculate of an individual. Such methods include obtaining ejaculate from the individual; allowing time for the liquefication of the ejaculate; separating the seminal plasma from the whole ejaculate; releasing the protein bound zinc from the seminal plasma; contacting the seminal plasma thus obtained with the device described herein; waiting for a color change reaction; and comparing the color change to a reference chart.

Additional objects, advantages, and novel features of this invention will become apparent to those skilled in the art upon examination of the following examples thereof, which are not intended to be limiting.

### EXAMPLES

### General Methods

### Measurement of Zinc Using apoCA-ABDN Via Fluorescence Ratiometric Methods

### Analysis of Free Zinc

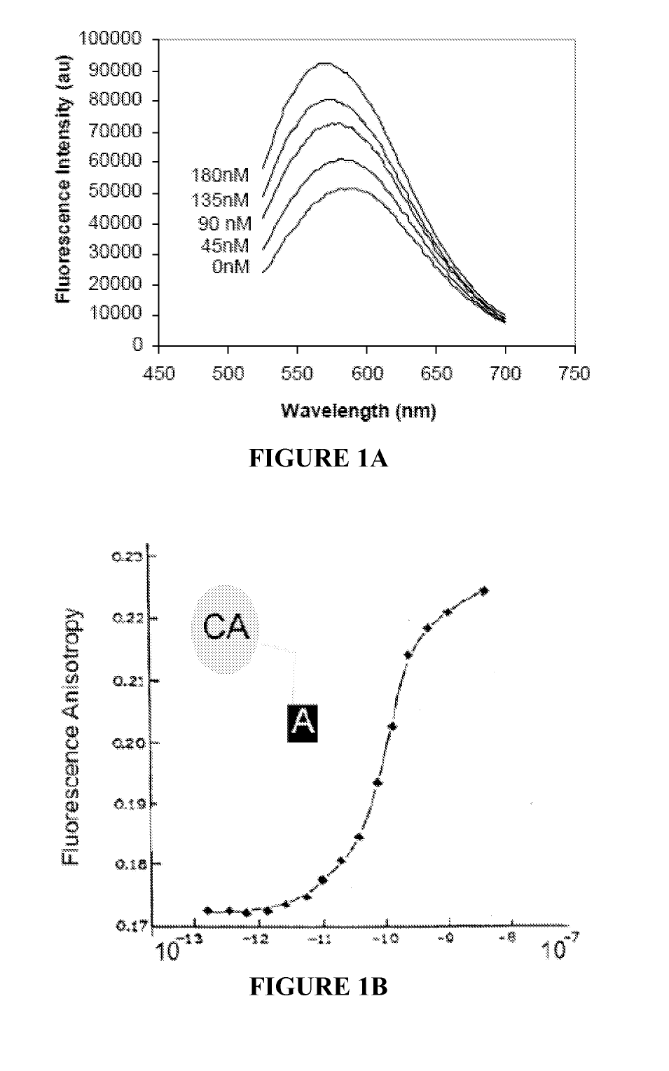

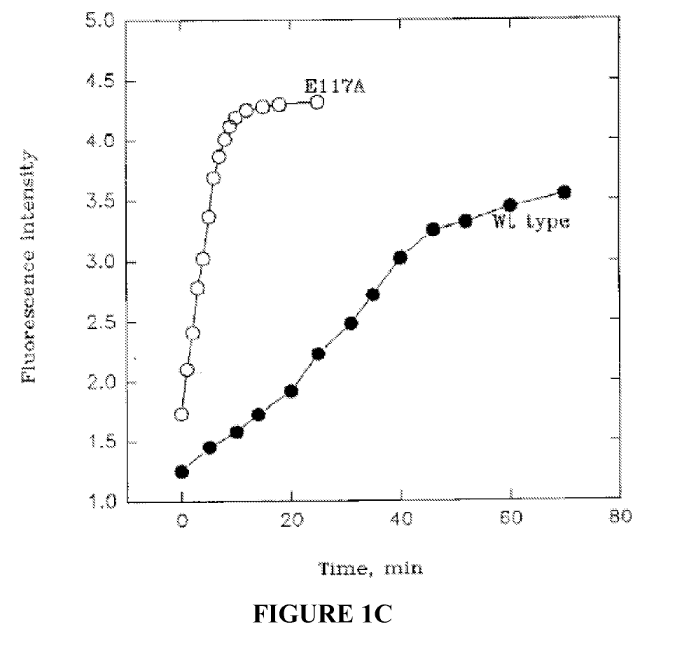

This Example illustrates using carbonic anhydrase (CA) as the zinc detector and either ABDN or dansylamide as the fluorescent reporter for determining the zinc level. In operation, the fluorescent reporter binds to the CA when the CA has a zinc in the “pocket”, i.e., holoCA. Upon binding to the holoCA, the reporter undergoes an increase in intensity and blue-shift in wavelength of the emission (FIG. 1A), as well as a change in fluorescence anisotropy (FIG. 1B). By starting with the apoCA, one then adds a test solution, and monitors the fraction of the reporter that is blue-shifted, or anisotropy-shifted, by the occurrence of zinc binding to the apoCA (FIG. 1A). The wavelength and anisotropy ratio measurements can be done in test tube or by confocal microscope. An entire family of genetically-engineered CA proteins with different affinities for zinc can be generated (FIG. 1C). By simply performing a competition assay with these different CA mutants, the binding strength of zinc to different ligands in ejaculate can be measured.

### Method for Fractionation of Semen Components

All containers, reagents and materials were cleaned of zinc by ion exchange, soaking in hot acid or hot EDTA, which chelates Zn²⁺, multiple rinses in 18 Mohm water, as appropriate. The success of all cleaning methods was verified by testing each procedure for the “blank” zinc contaminant level. Surfaces, e.g., soft glass, which are known to adsorb or release large amounts of zinc from solution are avoided.

Fresh ejaculate collected in tubes certified to neither adsorb zinc from samples nor to contaminate them within the limits of detection, i.e., femtogram, 10⁻¹⁵ g, was incubated at room temperature for 20 minutes to allow liquefaction. The samples were then diluted with one volume of 200 mM sucrose, 2.4 mM MgCl₂ and centrifuged at 400×g to remove intact sperm cells. The supernatant was stored at −80° C. for subsequent analysis, with freeze-thaw damage to proteins minimized.

Seminal plasma proteins were separated by size exclusion chromatography run at 4° C. The seminal plasma samples were diluted to a protein concentration of about 1 mg/mL in 150 mM NaCl and 100 mM sodium phosphate buffer (pH 7.1, buffer A). Up to about 5 mL of the resulting solution was then filtered through a 0.45 μm low protein-binding filter. The diluted seminal plasma samples (2-3 mL) were then applied to a 30 cm Sephacryl S300 HR column having a resolution range of 10 to 1500 kDa (Amersham Pharmacia Biotech). The mobile phase was buffer A, delivered at a flow rate of 1 mL/min via a peristaltic pump (Gilson) and 1 mL fractions were collected. Total protein in the eluted fractions was determined spectrophotometrically by 214 nm absorbance.

Total zinc content, i.e., free plus bound, of each semen component fraction was then determined by stable isotope dilution mass spectrometry and free zinc was determined by the apoCA fluorimetric method described above. The latter method for free zinc level determination is a fluorescence ratiometric method in which a fluorescent reporter molecule such as ABDN binds to a zinc sensor molecule, the metalloenzyme carbonic anhydrase, CA, when the CA has a zinc in the “pocket.” The zinc-containing holoenzyme increases the fluorescence of the reporter.

ApoCA was prepared by removing the Zn²⁺ with dipicolinate and dialysis against a zinc chelator. The apoCA was then mixed with the fluorescent reporter, both at 2 mM, in 50 mM HEPES-buffer. When there is no detectable Zn²⁺ in the fraction, i.e., less than the femtogram detection limit, the apoCA remains without zinc and does not bind to the fluorescent reporter, which emits its native fluorescence. When Zn²⁺ is present in the fraction, it binds stoichiometrically to the CA (K_{D} of 4 pM).

The resulting holoCA binds to the reporter, causing a shift in its emission wavelength from 600 nm to 560 nm and about an 8-fold increase in emission intensity. This system readily measures zinc in fluids from pM levels up. For Zn²⁺ levels well above the K_{D}, for example, low ∥M levels, the percent-occupancy approach is used in which the upper limit of the fluorescence sensitivity is set by the concentration of apoCA used and the lower limit is about 1% of that. For example, with 100 μM of apoCA and 100 μM of ABDN, the fluorescence shift will be maximal at 100 μM Zn²⁺ and is just detectable at about 0.1 to 1.0 μm.

The chromatography column is calibrated regularly with molecular weight standards (Sigma) and a parallel, calibrated column is used to resolve zinc-containing CA II (Sigma) to demonstrate efficacy of fractional zinc determination. Because carbonic anhydrase is the basis for the free zinc assay, the use of carbonic anhydrase holoenzyme with zinc and carbonic anhydrase apoenzyme with zinc removed provides an internal reference for total zinc as a fraction of total protein.

### Measurement of Zinc in Fluids

To measure zinc in a particular fluid, such as the semen plasma or prostatic fluid, including post prostate massage expressed prostatic fluid, one starts with an apoCA-ABDN solution at about 10 times the expected zinc concentration. An aliquot of plasma is added and a fluorescence spectrum is obtained. The magnitude of the emission peak shift relative to a control sample is observed. By appropriate dilution of the unknown, one then brings the sample into the right zinc concentration range for the final spectrum.

Calibration curves are run by the method of standard additions, using the matrix, e.g., seminal plasma, as the vehicle and adding zinc. Zinc chelators such as calcium EDTA are used to quench the fluorescence in order to verify that the emission shift is indeed due to zinc. SIDMS verifies the final concentration of zinc bound to the carbonic anhydrase after the carbonic anhydrase is isolated by dialysis, providing a verification of the accuracy of the method.

Access to an entire family of genetically engineered carbonic anhydrase proteins having a range of affinities for zinc would allow measurement of the binding strength of zinc to different ligands in ejaculate by simply competing for the zinc with the different carbonic anhydrase mutants.

### Example 1

### Measurement of Free Zinc that is not in Solution

To measure free zinc in material that is not in solution, such as in the cytosol of individual spermatozoa or in seminal globular proteins, microspectrofluorimetric methods for measuring zinc in brain tissue were used. Briefly, in this method, the material was stained to show the zinc pool of interest. Extracellular zinc, such as zinc on the outer surfaces of spermatozoa or zinc loosely coordinated with globular proteins, was stained with either cell-impermeable Newport Green, or by TSQ. Each stain has its particular strengths and weaknesses in this application. The material was stained, smeared on slides and the fluorescence was quantified in a fluorescence microscope and quantitative images captured on a laser-scanned confocal instrument and a cooled CCD camera (data not shown).

The distribution of total zinc in the different regions of the prostate gland and in different components, e.g., globular proteins and spermatozoa, of dried whole ejaculate can be determined by Synchrotron-induced X-ray fluorescence of zinc. The distribution of free zinc can be determined by histoanalytical methods specific to the subcellular localization of the zinc.

### Example 2

### Methods for Measuring Total Zinc in Prostatic Fluid

### Stable Isotope Dilution Mass Spectrometry (SIDMS)

Ejaculate, zinc-containing tissue or other samples, e.g., but not limited to, seminal plasma, prostatic fluid, including post prostate massage expressed prostatic fluid, or specific protein fractions are collected in tubes certified to neither remove zinc from samples by absorption or adsorption nor contaminate the samples within the limits of detection. Because semen has about 1000-fold more zinc than any other biological fluid, contamination will be less of a problem than usual in this type of work.

The samples are spiked with a measured amount of ⁶⁴Zn or ⁶⁶Zn before subjected to dissolution procedures to reduce them to elemental composition. All reagents are double-distilled in the laboratory in quartz stills, and made using ultrapure grade materials and 18 MOhm or better grade de-ionized water. Sample contact surfaces are all TFE (Teflon®), polypropylene or quartz.

Sample preparation after spiking generally progresses by (i) lyophilization; (ii) weighing; (iii) dissolution to elemental composition in concentrated hot nitric acid or perchloric; (iv) purification of zinc by ion exchange; (v) determination of ^{66/64}Zn ratio in the Isotope ratio Mass Spectrometer; and (vi) calculation of initial zinc concentration in the sample.

The accuracy of the final measure of zinc concentration generally depends on the degree of contamination or loss of zinc during sample preparation. Typically, in order to obtain a coefficient of variance of 5%, a minimum of 18 ng of zinc per sample is used. Given that all soft tissue has at least 60 ppm (dry) of zinc, this means no more than about 300 μg of tissue needs to be analyzed for 5% coefficient of variance.

### Flame Atomic Absorption Spectrophotometry (AAS)

Analyses of total zinc were performed in duplicate using AAS (PerkinElmer 5100 instrument). For sample preparation 10 μL-aliquots of semen plasma supernatant were mixed with 2990 μL of 0.5 M HNO₃ (OmniTrace Ultra, Merck) and incubated in closed test tubes for about 2 h at 60° C. Operating parameters were air/acetylene flame, 213.9 nm zinc line with deuterium lamp background correction. Zinc standards (Sigma-Aldrich) were 1000 mg/L and were diluted in 0.5 M HNO₃. Calibration curves down to the range of 0.05 μg were routinely obtained during sample analysis and were quite linear.

### Example 3

### Free and Total Zinc Analysis in Prostatic Fluid

### Normal Distribution

Frozen human semen from 3 young men (sperm donors) and from 15 men with prostate symptoms (no biopsy was necessary or biopsy-confirmed BPH) was liquefied at 37° C. for 30 min. The samples were centrifuged at 1000×g for 10 min to separate spermatozoa from the seminal plasma. Free zinc was measured spectrophotometrically in the seminal plasma by adding 10 μL of seminal plasma to 990 μL of Zincon (extinction coefficient of the Zn:Zincon at 620 nm; 17,500 M⁻¹ cm⁻¹). This procedure gave a working range of approximately 1 μM to 100 μM in the stoichiometric assay mode. To measure total zinc by FAA, 10 μL of seminal plasma samples were diluted into 1810 μL of 0.5 M HNO3 and analyzed for total zinc by standard methods. Two measurements were made for each sample.

Total zinc in the seminal plasma was about 3.5 mM (range 3-6 mM). The concentration of free zinc averaged about 0.4-0.5 mM, as measured after dilution into HEPES at 7.4 as referred back to the undiluted sample. The 0.4 mM value of free zinc is about 400,000-fold higher than that found in most extracellular fluids and the 3.5 mM value of total zinc is about 20-fold higher than most soft tissue.

### Distribution of Free and Total Zinc Among Pools of Zinc in Ejaculate and Seminal Plasma

17 men aged 42 and older and presenting symptoms of prostatitis or prostate enlargement or malfunction provided ejaculate samples collected at home. A sample kit with a unique identification number consisted of a collection vial, cold shipment container and instructions for collection of the ejaculate sample at home. The unique identification number was used to identify the samples and to correlate the data obtained with pertinent information regarding the participant's prostate health.

Sample preparation was as described for those obtained from normal men except that the 200 μL of the seminal plasma was subjected to size-exclusion fractionation into 42 fractions (500 μL) on a Sephadex 0 column and the free zinc and protein concentration were then analyzed for each fraction. Free zinc was measured after dilution of 10 μL of each fraction into 90 μL of Zincon solution, as described above. Total protein and peptide concentrations were measured with a micro BCA protein assay kit (Pierce Biotechnology). 20 μL of each seminal plasma aliquot was mixed with 280 μL of 20 mM Tris-HCl buffer, pH 7.4 and 200 μL of assay reagent. The solutions were incubated at 60° C. and absorbance was measured at 562 nm.

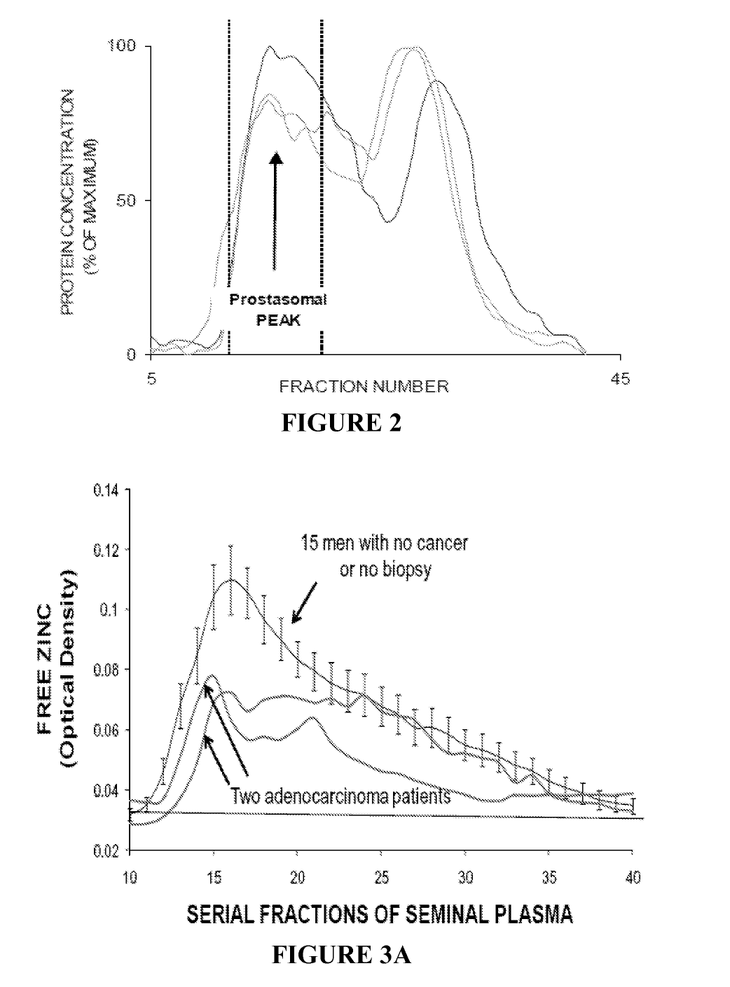

FIG. 2 shows that the seminal protein has two distinct peaks, one early peak that corresponds to the high molecular weight (HMW) proteins and one later peak that corresponds to the low molecular weight (LMW) peak. The HMW peak was confirmed to be highly enriched in the giant globules of prostate-secreted proteins, prostasomes. Thus, the free and total zinc that was measured from this prostasomal fraction represents the zinc in prostatic fluid per se. The free zinc, which is emblematic of prostatic secretion, was highly enriched in the prostasomal fraction.

### Seminal Zinc is Reduced in Gleason Stage 6-8 Tumors

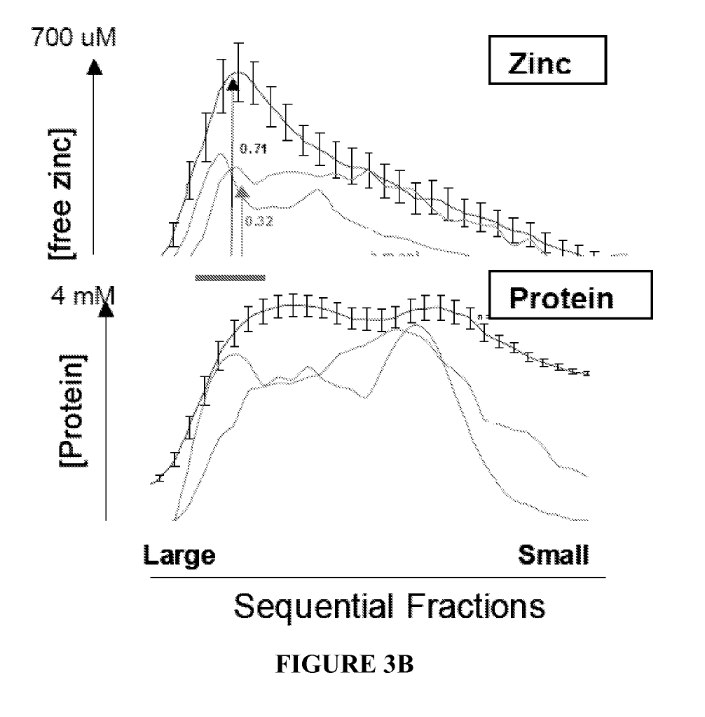

Analysis of the protein and zinc content of seminal plasma demonstrated that men with prostate cancer, confirmed by biopsy, have measurably lower protein and zinc in the prostasomal fraction of seminal fluid. FIGS. 3A and 3B show the total zinc and protein, respectively, measured in each fraction for 15 “normal” men (lines with range bars) and 2 men with prostate tumors (individual lines). As can be seen, the total protein measured in the seminal plasma of the “normal” men displayed the two peaks discussed above, the HMW “prostasomal fraction” and the LMW peak. The peaks were less distinct in the pooled data because fraction numbers were not adjusted to “synchronize” the first peak. The two men with confirmed prostate cancer also had the LMW protein concentration peak but the prostasomal protein fraction peak was essentially absent.

The free zinc in the prostasomal fraction also was markedly lower in both men with cancer (FIG. 3A) and was no more than 50% of the control value. Translating the absorbance measurements to actual concentrations of free zinc, the 0.71 absorbance (baseline subtracted) is equal to 2 micromolar in the cuvette; correcting for the dilution (1000-fold) this gives a peak concentration of the free zinc in the prostasomal fraction of 2 mM in the healthy men and less than half, i.e., 1 mM, for the two cancer patients.

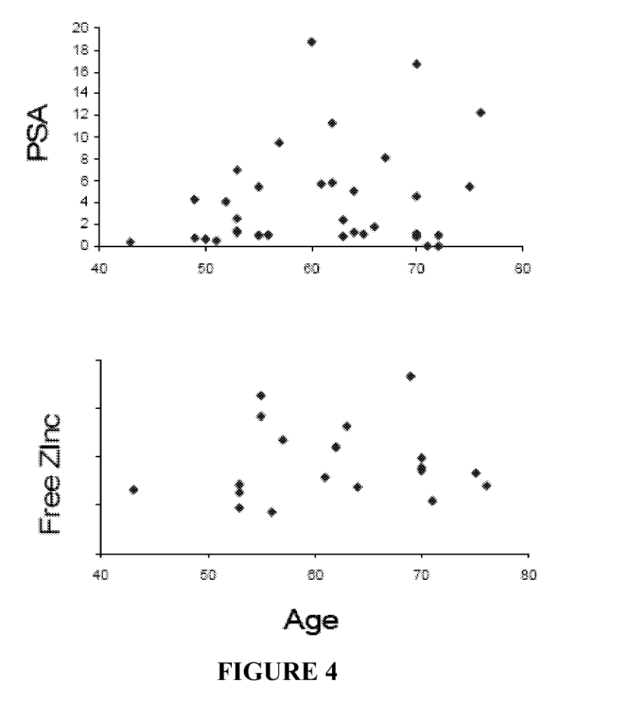

In comparing PSA with age (FIG. 4, top), only the slightest trend of PSA increasing with age was seen because the men who would typically have very low PSA (under 40) have not been tested. To evaluate whether prostasomal zinc varies with age (FIG. 4, bottom) or PSA, the average peak concentration of zinc in the prostasomal fraction was calculated. No significant correlation was found between prostasomal free zinc and either age or PSA. This indicates that the zinc data are a predictor of prostate cancer independent of PSA. The correlation between total zinc concentration in seminal plasma and the concentration of free zinc in the prostasomal fraction was essentially zero (r=0.003) indicating that the two measures are not simply redundant estimates of the prostate function.

### Example 4

### Histochemical Imaging of Prostate

The tissues to be used in this work include prostates harvested from normal men who died without any prostate disease and prostates harvested by prostatectomy or by autopsy from men who had confirmed aggressive prostate cancer. The tissues are frozen without fixative within an 8-hour postmortem interval. This can include tissues in existing tissue banks, so long as the tissue is frozen without fixative within 0-8 hours postmortem.

### Tissue Distribution of Total Zinc by Synchrotron-Induced X Ray Fluorescence Imaging

Frozen sections are cut and mounted on glass slides and on mylar slides. The glass-mounted tissue is fixed over aldehyde vapor, then in aldehyde solution for conventional immunostaining to identify various cytoarchitectonic regions. The mylar-mounted sections are sealed in dust-free containers and processed by synchrotron-induced X Ray fluorescence imaging.

### Distribution of Free Zinc at the Macroscopic and Light Microscopic Level

Fresh-frozen tissue sections are stained with either TSQ or Newport Green (cell permeable) or Zinpyr for imaging of the intracellular zinc pools. Different stains show different “pools” of zinc in the tissue. Thus, the lipophilic stains (TSQ and Zinpyr) readily stain zinc that is sequestered in the secretory granules or zincosomes in which it is most highly concentrated. Newport green and apoCA-ABDN, on the other hand, stain cytoplasmic zinc but cannot penetrate these zincosomes and does not stain those cell compartments. Thus, comparison of the differences in staining indicate subcellular localization of zinc.

### Localization of Zinc at the High-Magnification Light and Electron-Microscopic Level

The silver methods of Danscher is used. For the silver staining or autometalography (AMG), the tissue is sectioned frozen, then exposed to sulphide vapor (HS) while kept frozen. This treatment precipitates zinc as ZnS in the frozen tissue, thus immobilizing it in situ in whatever subcellular organelles it happens to be. After sulphide precipitation, the tissue is fixed by further exposure to aldehyde vapor (still frozen) before conventionally fixed by aldehyde immersion. Next the tissue sections are developed in a silver developer solution in which the ZnS crystals catalyze reduction of silver, forming silver nanoparticles around the ZnS. Developed sections are then either counter-stained, cleared, and cover-slipped for light microscope analysis; or dehydrated, embedded in plastic, and ultratomed for analysis in electron microscope.

### Example 5

The distribution of zinc in ejaculate and prostatic fluid were characterized and validated using atomic absorption (AA) and X-Ray fluorescence. Experiments have shown that fluorescent imaging can be used for visualizing the level of zinc in the zinc-sequestering and secreting portions of prostatic tissue.

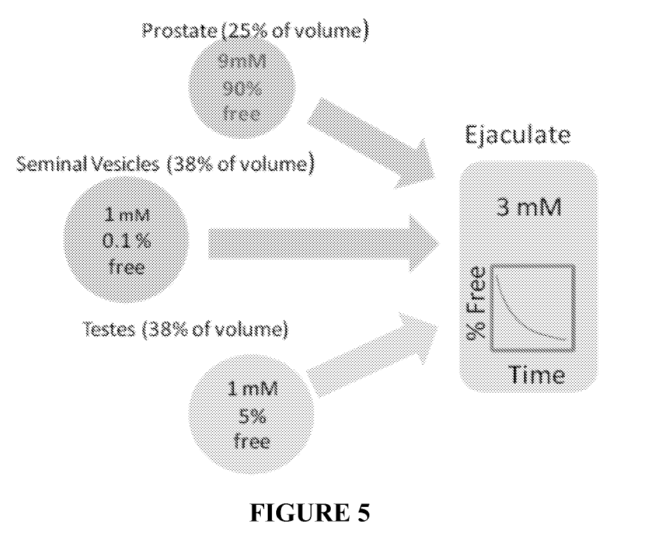

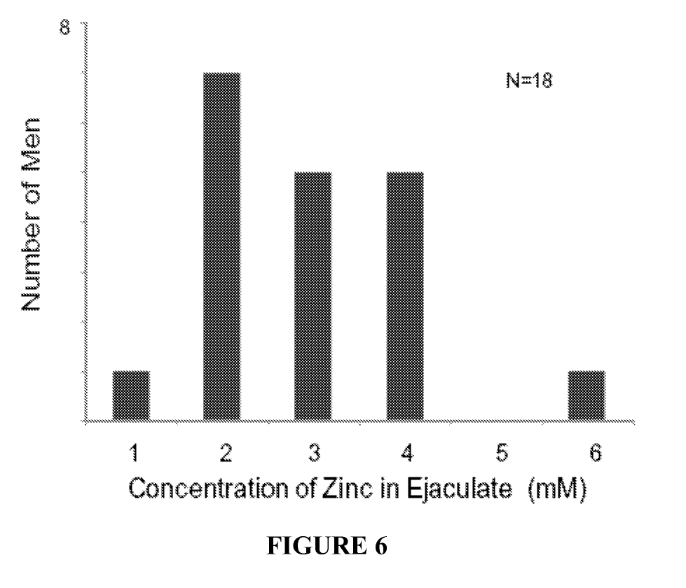

FIG. 5 shows an overview of the distribution and speciation of zinc in prostatic fluid and in ejaculate. The total amount of zinc in the ejaculate of 18 men with no known cancer is shown in FIG. 6. This example included 15 elderly men who had reported for prostate exams and judged to be tumor free in addition to 3 men who were donating sperm. The frequency histogram showed that 3 mM is the approximate mean and 2 mM the mode of the distribution of total zinc in ejaculate.

The distribution of zinc amongst the various fractions of seminal plasma was also examined, and have found that there are two main “pools” of zinc in seminal plasma, and that the distribution of zinc between the two pools changes over time as plasma (or ejaculate) is allowed to stand at room or body temperature. This change is shown schematically in the diagram of zinc speciation in FIG. 5, which shows that the percentage of free zinc declined over time.

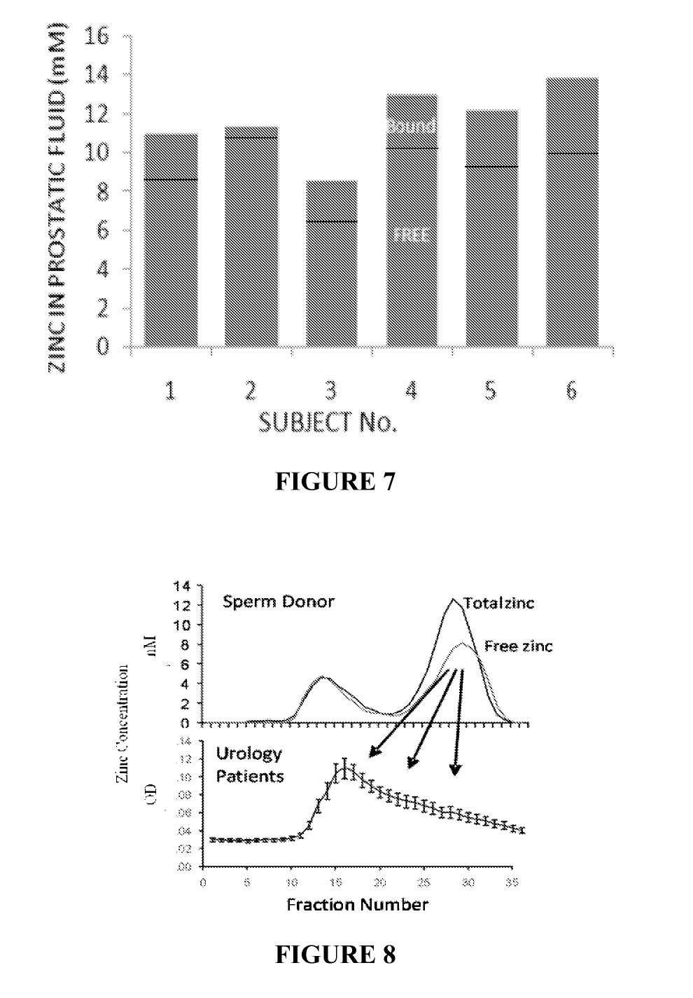

The sequence of events is as follows. In prostatic fluid, most of the “free” zinc is weakly coordinated, for example, with 100 mM of citrate (Zn:Cit K_{D} about 10 mM). This is shown in data for 6 men in which the free zinc was measured with a pZn Meter and the total zinc with AA (FIG. 7). A second major zinc-binding ligand in prostatic fluid is PSA, which binds zinc moderately (K_{D} about 50 μM). Typical PSA concentrations in prostatic fluid are about 2 mM.

However, when prostatic fluid mixes with the fluids from the seminal vesicles and from the testicles, it is believed that the distribution of zinc begins to change relatively quickly. This is because albumin and semenogelin proteins from the seminal vesicles have extensive zinc-binding capacity. Semenogelins I and II both have 8-10 zinc binding sites with K_{D} about 1 μM. This interaction, in which PSA is activated via the chelation of zinc by the semenogelins, which are then cleaved by the PSA, is believed to be what underlies the “liquefaction” of the coagulum in ejaculate. It is believed that the binding sites are preserved after the semenogelins are cleaved into fragments by PSA.

The influence of this rather slow (tens of minutes) chelation of the zinc away from the PSA and the citrate and onto the semenogelins (and albumin) for detecting prostate cancer is that in prostatic fluid, or in freshly-expressed ejaculate, the zinc is mostly (˜80%) “free” (i.e., coordinated with citrate). However, over the ensuing 15-60 minutes (depending on the temperature at which the ejaculate is kept) the zinc becomes bound to the semenogelins and albumin. But the binding of zinc does not occur with prostatic fluid.

In one case, starting with ejaculate samples that had been flash-frozen immediately after expression (from a fertility clinic) samples of the seminal plasma (basically the supernatant fluid obtained after a brief liquefaction and centrifugation) was taken. When these samples were ran through a size—exclusion column and the zinc associated with the different proteins were measured, using a sample from a single subject (one shown in the FIG. 6) two peaks were found: one associated with the largest proteins (presumably semenogelins) and the other, associated with the smallest ligands, presumably citrate (FIG. 8). FIG. 8 is a graph showing “free” (weakly bound) zinc in successive protein fractions of seminal plasma. Note in the top panel (single subject) that there are two peaks of zinc, one at fraction 15 (large proteins) and one at fraction 30 (small ligands). In the lower panel, the small-ligand associated peak is gone. The upper sample was flash frozen, the 15 samples in the lower panel were frozen slowly, after expression, collection, and placement in −18 ° C. freezers.

When semen that men expressed at home, and then put in a container, which they placed in their home freezers, was used it was found that after liquefaction and centrifugation, the seminal plasma (supernatant) still showed a zinc peak associated with the high molecular peak, but the zinc associated with the small ligand was substantially reduced.

### Example 6

Ejaculate samples were collected from 18 men who were getting prostate examinations. After liquefaction and centrifugation, the supernatant was applied to the protein separation column and the free zinc that co-eluted with each fraction was measured.

Two of the 18 men had high-grade (Gleason 7-8 or higher) tumors, which were relatively large in volume (late stage), whereas 17 of the men either had no cancer at biopsy or were not judged in need of biopsy. The free zinc in the fraction of the seminal plasma corresponding to the large proteins (i.e., peak 1 in the zinc profile) was reduced by 50% or more in both of the adenocarcinoma patients (FIG. 3A).

A third man was found, upon biopsy, to have no tumors on one side of the prostate and only a very small, low-grade (Gleason 2-3) tumor on the other. His zinc profile was intermediate between the “normals” and the “cancer” groups.

### Example 7

This example illustrates methods for using prostatic fluid for determining the free zinc level.

The present inventor has found that the zinc secreted from the prostate could be studied much more easily and accurately by looking directly at the prostatic fluid per se. After mixing with seminal and testicular fluid, the zinc in the prostatic fluid is diluted and changes its binding, as discussed above.

The free zinc levels were determined in 10 samples of prostatic fluid that were collected during prostate massage from men who were receiving routine prostate examinations. The free zinc in nine of the fluid samples were all grouped fairly closely around a mean value of 8.5 mM (SD=2.5), but the 10th man had only about 5% of that zinc, namely about 0.5 mM.

Upon checking, it was found that the man in question had a PSA of 6.2, and has had two prior biopsies due to the combination of his high PSA and DRE results, which suggested a tumor. Therefore, while the zinc value for this man is shown, that value was not included in the “normal” subjects. Rest of the men never had been recommended for biopsy.

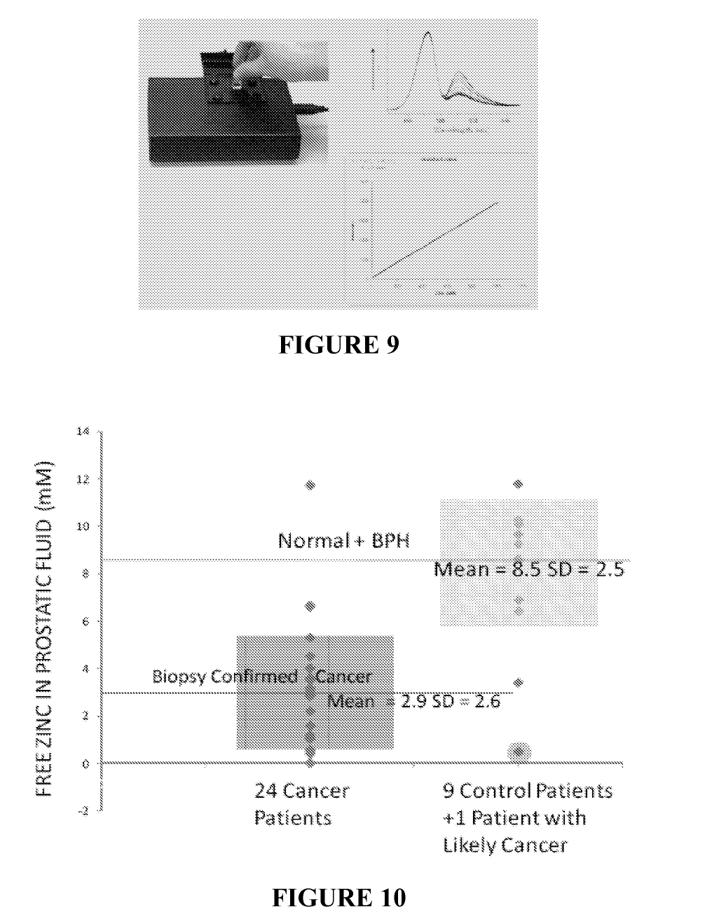

The device (“pZn meter”) shown in FIG. 9 was used to measure the free zinc in 24 samples of expressed prostatic fluid provided by the Northwestern SPORE. These samples were expressed from the prostate gland after prostatectomy, so the conditions of the prostate “massage” were not identical in the cancer patients (ex vivo massage) and in the control patients (in vivo massage). This notwithstanding, it is noteworthy that the concentration of free zinc in the cancerous glands was about ⅓ as high as from the normal glands, and that there was almost no overlap in the zinc concentrations for the two groups (FIG. 10). Further, it can be seen that 5 of the zinc concentrations in the cancer group were below 1 mM, i.e., less that ⅛th of the control mean value.

Referring again to FIG. 9, the device can be connected to a computer, for example, by the USB port shown on the right side. In this particular embodiment, the computer is used for data processing and provides power for the LEDs. In use, the cover is closed and the fluorescence is measured to determine the zinc level. A sample fluorescence spectrum and calibration curve are also shown in FIG. 9. Typically, measurement of the free zinc in the prostatic fluid is done after dilution (generally 1:3000 and 1:6000) into cuvettes with 50 mM HEPES (pH 7.4). The cuvette is then placed in the pZn meter, fluorescent probe for zinc is added, and the concentration of the free zinc is measured by fluorimetry. Both the 1:3000 and the 1:6000 dilutions are measured, as replicates. Calibration standards are run before and after each measurement of prostatic fluid.

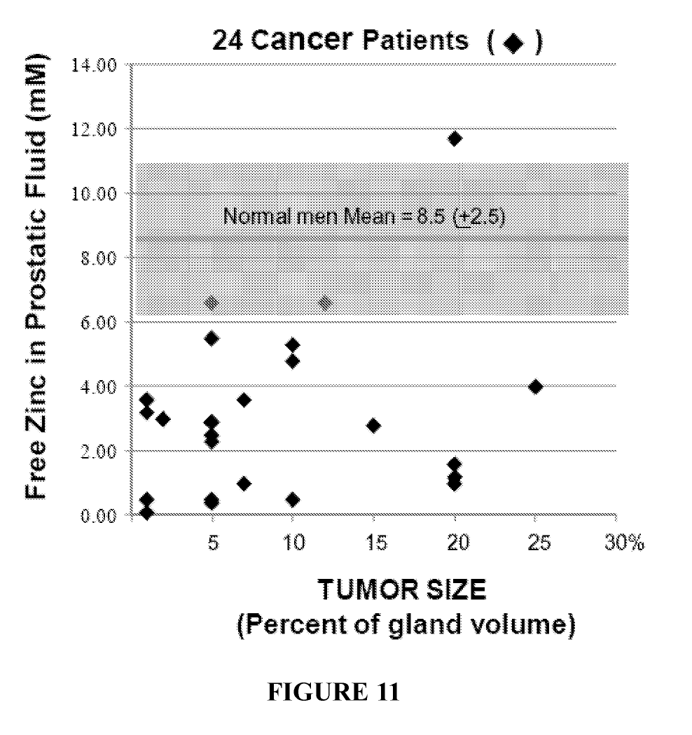

The concentration of zinc in the prostatic fluid in 24 cancer patients were compared with the tumor size. As can be seen in FIG. 11, the concentration of zinc in the prostatic fluid did not vary with tumor size (tumor volume as percent of total volume). However, even the smallest tumors (1 to 5% volume) were accompanied by some of the lowest concentrations of zinc (FIG. 11). Comparison of prostatic zinc concentrations with the stage of tumors (Gleason Scores), the patient's age, PSA, and the overall size of the gland (in grams) showed that none of those variables was significantly correlated with the prostatic zinc concentrations for the 24 patients (data not shown).

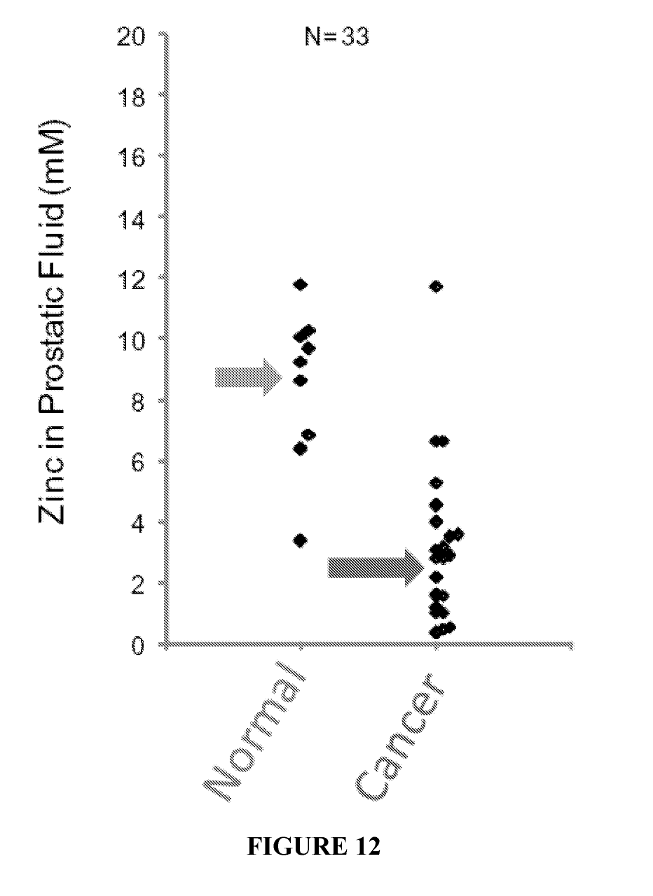

The mean free zinc concentration for normal men was 8.5 mM. See FIG. 12. As FIG. 12 shows, it is clear that there is a cancer related drop in the zinc content of prostatic fluid. In FIG. 12, the one “normal” subject with low zinc level (˜3 mM) had a PSA of 8+ and has had no biopsy.

The Receiver Operating Curve analysis (for detection of adenocarcinoma of any grade), it was observed that 95% confidence limits for Sensitivity (0.75 to 1.0) and Specificity (0.86 to 0.99). All zinc studies have yielded AROC results that are better than typical PSA results.

### Example 8

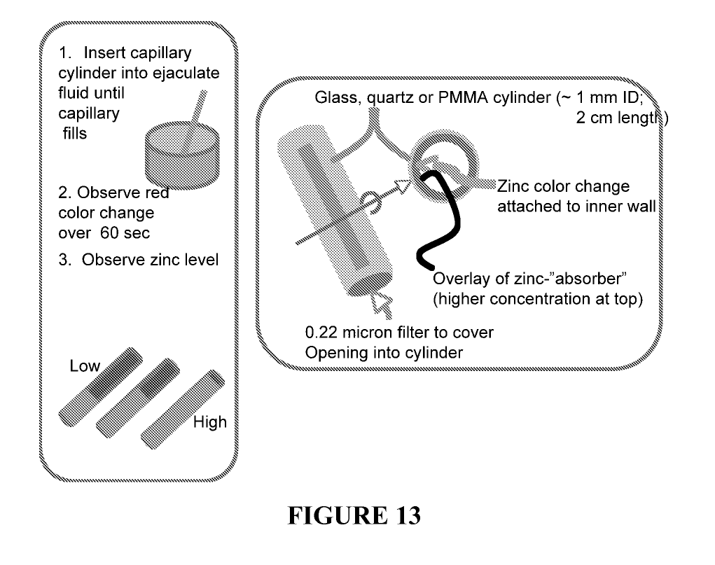

FIG. 13 shows another embodiment of the device that can be used to test zinc level in a fluid sample. In this embodiment, a zinc-binding molecule that changes color when bound to zinc is attached to the inner surface of a capillary tube, and a filter (e.g., 0.22 micron pore size) covers the entrance to the tube. When dipped into a fluid sample (e.g., liquefied ejaculate), the capillary action (i.e., surface tension) of the tube allows the tube to be filled with the fluid. The left panels outlines the three steps of zinc determination: (1) filling the capillary, (2) waiting for the color change reaction; and (3) comparing the color change to a reference chart. In this manner, one can readily determine the zinc level in a fluid sample.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method for screening a subject for the presence or elevated risk of developing prostate cancer comprising:
measuring a level of free zinc in a seminal or prostatic sample of the subject; and
comparing the measured zinc level with a control zinc level to screen the subject for the presence or elevated risk of developing prostate cancer,
wherein when the seminal sample of the subject is used, said step of measuring the level of free zinc comprises:
subjecting the sample to the free zinc level measuring step within 5 minutes or less of the time the sample leaves the subject's body; or
using the measured level of free zinc to determine the level of free zinc at the time the sample leaves the subject's body.

2. The method of claim 1, wherein the control zinc level comprises the level of free zinc in the normal individual.

3. The method of claim 1, wherein the level of free zinc in the fluid is measured optically.

4. The method of claim 1, wherein prior to said step of measuring the free zinc level a reagent is added to the sample, wherein the reagent is capable of releasing zinc from citrate.

5. The method of claim 3, wherein said method of measuring the free zinc optically comprises:
contacting the sample to a zinc-binding moiety under conditions sufficient to bind the free zinc to the zinc-binding moiety, wherein the zinc-binding molecule has a different optical property when bound to zinc relative to its non-zinc bound state;
determining the optical property of the zinc-binding molecule; and
correlating the optical property of the zinc-binding molecule with the level of free zinc in the fluid.

6. The method of claim 5, wherein the zinc-binding moiety comprises a chromophore or a fluorophore.

7. A method for determining the presence or elevated risk of developing prostate cancer in a subject, said method comprising:
determining a level of free zinc in a seminal or prostatic sample of the subject; and
correlating the level of free zinc to the presence of prostate cancer or elevated risk of developing prostate cancer in the subject,
wherein when the seminal sample of the subject is used, said step of determining the free zinc level comprises:
subjecting the sample to the free zinc level determination process within 5 minutes or less of the time the sample leaves the subject's body; or
using the determined level of free zinc to determine the level of free zinc at the time the sample leaves the subject's body.

8. The method of claim 7, wherein when the level of free zinc is determined optically.

9. The method of claim 8, wherein said method of determining the level of free zinc comprises:
contacting the sample to a zinc-binding molecule under conditions sufficient to bind the free zinc to the zinc-binding moiety, wherein the zinc-binding molecule has a different optical property when bound to zinc relative to its non-zinc bound state; and
correlating the optical property of the zinc-binding molecule to the level of free zinc in the sample.

10. The method of claim 8, wherein the zinc-binding molecule comprises a chromophore or a fluorophore.

11. A device for visually determining a zinc level in a bodily fluid, said device comprising:
a zinc-binding molecule that allows optical determination of the zinc level in a bodily fluid sample;
means for confining the zinc-binding molecule to a region in space; and
a surface effective for visually observing optical change of said zinc-binding molecule within the region thereby allowing optical determination of the zinc level.

12. The device of claim 11 further comprising a reagent that releases zinc from a protein in said bodily fluid.

13. The device of claim 12, wherein said reagent is a pH lowering reagent, diethyl pyrocarbonate, cystine diethyl pyrocarbonate residue, a protease, a zinc-chelating reagent with zinc binding affinity of at least 1 mM, or a combination thereof.

14. The device of claim 11, wherein said zinc-binding molecule has a different optical property when bound to zinc relative to its non-zinc bound state.

15. The device of claim 11, wherein said zinc-binding molecule is confined to the defined region via covalent binding to a solid substrate.

16. The device of claim 11, wherein said zinc-binding molecule is retained in the defined region due to the partition co-efficient of the molecule.

17. The device of claim 11 further comprising an interface that separates a bodily fluid collection region from the zinc-binding molecule.

18. The device of claim 17, wherein said interface allows selective permeation of zinc ions to reach the region containing the zinc-binding molecule.

19. The device of claim 18, wherein said selective permeation is due to size, solubility, charge, or other physical properties.

20. A kit for determining the zinc level in a bodily fluid of an individual comprising:
a device of claim 11; and
a reference chart.

21. The kit of claim 20 further comprising a container for collecting the bodily fluid.

22. The kit of claim 20, wherein said reference chart is a zinc level color chart.

23. The kit of claim 22, wherein the zinc level color chart designates a specific color for low, normal and high levels of zinc.

24. A method of optically determining a zinc level in a bodily fluid of an individual comprising:
contacting the bodily fluid sample obtained from the individual with a zinc-binding molecule, wherein the zinc-binding molecule has a different optical property when bound to zinc relative to its non-zinc bound state; and
observing the optical property of the zinc-binding molecule, thereby determining the zinc level in the bodily fluid of the individual.

25. The method of claim 24, wherein the zinc-binding molecule is bound to a solid substrate.

26. The method of claim 24, wherein zinc is separated from at least a portion of the bodily fluid sample prior to contacting with the zinc-binding molecule.

27. The method of claim 24, wherein the optical property of the zinc-binding molecule is chromophore or fluorophore.

28. The method of claim 24, wherein said step of determining the zinc level comprises visually comparing the optical property of the zinc-binding molecule with a reference chart.
